(19) United States
(12) Patent Application Publication
(10) Pub. No.: US 2006/0189516 A1
(43) Pub. Date: Aug. 24, 2006

(54) Method for producing cross-linked hyaluronic acid-protein bio-composites

(75) Inventors: Chiung-Lin Yang, Taipei (TW); Jui-Hsiang Chen, Hsinchu (TW); Shiao-Wen Tsai, KaoHsiung Hsien (TW); Hsin-Nung Shih, Taipei City (TW); Lih-Yuann Shih, Taipei City (TW)

Correspondence Address: BRUCE H. TROXELL, SUITE 1404, 5205 LEESBURG PIKE, FALLS CHURCH, VA, 22041, US

(73) Assignee: Industrial Technology Research Institute

(21) Appl. No.: 11/208,596

(22) Filed: Aug. 23, 2005

Related U.S. Application Data
(63) Continuation-in-part of application No. 10/076,288, filed on Feb. 19, 2002, now abandoned.

(51) Int. Cl. A61K 38/16; C07K 14/47

(52) U.S. Cl. 514/8; 530/395

(57) ABSTRACT

This invention is concerned with a new method for producing cross-linked hyaluronic acid—protein bio-composites in various shapes. In the present process, a polysaccharide solution and a protein solution are mixed under moderate pH values in presence of salts to form a homogenous solution, which can be processed into various shapes, such as membrane, sponge, fiber, tube or micro-granular and so on. After then, the homogenous solution is subjected to a cross-linking reaction in organic solvent containing weak acid to produce an implantable bio composite material having excellent bio-compatibility, biodegradability, prolonged enzymatic degradation time, and good physical properties.

### RELATED APPLICATIONS

This application is a continuation-in-part of U.S. Ser. No. 10/076,288, filed on Feb. 19, 2002, entitled “A METHOD FOR PRODUCING CROSS-LINKED HYALURONIC ACID-PROTEIN BIO-COMPOSITES” and currently pending.

### BACKGROUND OF THE INVENTION

1. Field of Invention

This invention relates generally to a new method for producing cross-linked hyaluronic acid—protein bio-composites in various shapes, and in particular, to a method for producing cross-linked hyaluronic acid—protein bio-composites from a homogenous solution preparing by mixing hyaluronic acid and protein at various ratios. The prepared bio-composites can be processed into different shapes. The present invention also relates to the use of the bio-composite in prevention or reduction of post-surgical adhesion and in bone regeneration.

2. Description of the Related Art

Hyaluronic acid (HA) is a muco-polysaccharide occurring naturally in and purified from the vertebrate tissues and fluid, and having a linear structure with high molecular weight from several thousands to several millions daltons depending on its source and purification method. Karl Meyer et al. in 1934 first reported that HA contains glucuronic acid and glucosamine and was isolated and purified from the vitreous humor of cow. HA is a linear chain polymer having repeat units of N-acetyl-D-glucosamine and D-glucuronic acid residues bonded through beta (1→3) bonding and then beta (1→4) bonding. HA is widely distributed in connective tissues, mucous tissue, crystalline lens and capsules of some bacteria. In commercial applications, HA has been used as a matrix in drug delivery, an arthritic agent, a healing agent for arthritic operation or general wound healing. In industrial production. HA is mainly extracted and purified from the cockscomb, but HA can also be isolated and produced from the capsules of Streptococci spp. by fermentation bio-technique.

HA aqueous solution shows both a high viscosity and flexibility. HA is generally called visco-elastic matrix when applied in the ophthalmology. The viscoelastic characteristic is attributed to the sponge polymeric network formed from bulk molecular volume HA having high MW. HA is in vivo synthesized from HA synthetase that exists in the plasma membrane, and hydrolyzed by the hyaluronidase that exists in lysozyme. The interaction of HA and proteoglycans can stabilize the structure of resultant matrix and modify the behavior of cell surface. This characteristic exhibits many important physiological functions, including: lubrication, water-absorption, water retention, filtration, and can modulate the distribution of cytoplasmic protein.

It is known that HA possesses advantages of (1) naturally occurring in human body, (2) no immune reaction, (3) capable of being easily degraded and absorbed in human body, (4) easily obtainable, (5) being a high molecular weight bio material applied in medicine. The major application of HA is in the ophthalmic operation of cataract and cornea damage. High molecular of aqueous HA solution is injected into eye as a visco-elastic fluid to maintain the morphology and functions of eye. HA has been recently applied in wound healing, tissue anti-adhesion after surgery and drug delivery applications. HA is present in intra-cells as a complex with protein in tissue, which forms a jelly matrix owing to it high water retention and can thus be useful in cosmetic application as an anti-aging agent.

Collagen is a structure protein found in animals. It is a naturally occurred biopolymer, and its moiety causing an immune-reaction could be eliminated via isolation, purification and optional treatment with enzyme (such as pepsin), to give collagen having a good bio-compatibility. Collagen can be processed by various reconstruction, chemical cross-linking reaction and optional additional processing procedure to form into different shapes, such as plate, tube, sponge, powder or soft fabric. Since collagen will be biodegraded in vivo and is a low toxic polymer having excellent bio-compatibility in human body, it has been used as a hemostatic agent, nerve regenerating agent, tissue anaplastic agent, scald dressing material, hernia repair, urethra operation, drug delivery, ophthalmology, vaginal contraceptive, cardiac valve repair, blood vessel operation and operating structure, and other biomedical materials.

Gelatin is a denatured collagen and its amino acid content is similar to the collagen but different in structure and chemic-physical properties. Up to date, it has been used in a wide variety of food application and medical research, such as hemostatic cotton and drug delivery.

HA and collagen are the major components of extra-cellular matrix. Gelatin is also made from collagen. Therefore, gelatin also has good bio-compatibility and biodegradation in human body. The gelatin composites can also be used in the development of implant matrices in biomedical materials field, such as histological engineering, active ingredient releasing system or as materials for preventing tissue from sticking after surgery.

(1) Milena Rehakova et al., 1996, Journal of biomedical materials research, Vol., 30, pages 369-372, describes a method for preparing collagen and hyaluronic acid composite materials through the use of glyoxal and starch dialdehyde as a cross-linking agent. The collagen was dispersed in 0.5M acetic acid solution, and then HA was added to the solution and reacted for 5 minutes. Fiber was precipitated and filtered, washed several times with water and alcohol, and dried at a temperature of 35° C., and then a fiber structure in the form of a film having a smooth surface was produced. The cross-linking of the composite material was carried out in the presence of an aqueous starch dialdehyde solution. In the case of using glyoxal as the cross-linking agent, the cross-linking was carried out by adding HA and glyoxal to the suspension of collagen, or adding glyoxal to the suspension of collagen first and then adding HA.

(2) Jin-Wen Kuo et al., 1991, Bio-conjugate chemistry, Vol.,2, pages 232-241, describes a method for preparing water-insoluble derivatives of hyaluronic acid by reacting high molecular HA with 1-ethyl-3-(3-dimethylaminopropyl) carbodiimide at a pH of 4.75. In a general experiment, sodium hyaluronate was dissolved in distilled water to produce a 4 mg/ml HA solution. In some reactions, amine and sodium hyaluronate were added into the HA solution and mixed together. The pH of the aqueous solution was adjusted to pH 4.75. Carbodiimide was dissolved in either water or isopropanol, depending on the solubility of carbodiimide.

After mixing of HA and carbodiimide, the resultant solution was maintained at a pH of 4.75 by addition of 0.1N HCl using a pH meter. The reaction mixture was kept at room temperature for 2 hrs, then HCl solution was added until a concentration of HCl was 5% (w/v) in the solution, and then a precipitate is formed after adding 3 time volume solution of ethanol. Non-reacted chemical reagent was washed out for 2-3 times with distilled water. Finally, the precipitate was dissolved in de-ionized water before lyophilization.

(3) Lin-Shu Liu et al., 1999, Biomaterials, Vol., 20, pages 1097-1108, states a method for preparation of hyaluronate-polyaldehyde by treatment of hyaluronate with sodium periodate. Hyaluronate-polyaldehyde was prepared by oxidizing sodium hyaluronate with sodium periodate. A collagen-hyaluronate matrix was synthesized by covalent bonding of aldehyde group to the collagen to obtain a material for supporting cartilage tissue or repairing bone.

(4) D. Bakos et al., 1999, Biomaterials, Vol., 20, pages 191-195, describes a new method for preparing bio-composite material. The composite material consisted of nine parts by weight of inorganic component hydroxyapatite and one part by weight of organic component including 92wt % collagen and 8wt % hyaluronic acid. Hydroxyapatite particles were gradually added into the solution of hyaluronic acid in de-ionized water, and intensively stirred and mixed. Separately, very fine collagen fibers (1% by dry weight) were dispersed in de-ionized water after dry fibrillation of lyophilized fibers of collagen. The two prepared dispersions were mixed together to form complex precipitates. The precipitate was filtered and dried at a temperature of 37° C. to obtain a composite which did not undergo any cross-linking reaction.

(5) C. J. Doillon et al., 1988, Biomaterials, uses a porous sponge of collagen as a support for the growth of epithelium and fibroblast cell, and as a matrix of artificial skin. HA and/or fibronectin can enhance the repair of wounded skin and the proliferation of cell. These bulk molecules can modify the behavior of cell in tissue culture. The method for its preparation includes a step of dispersing water-insoluble collagen (1% by weight) in hydrochloric acid solution at a pH 3.0. In this step, 1% w/w of hyaluronic acid, fibronectin, dermatan sulfate and chondroitin-6-sulfate were added into the collagen solution. The dispersion solution was frozen at −30° C., and then lyophilized before cross-linking.

(6) S. Srivastava et al., 1990, Biomaterials, Vol., 11, pages 155-161, indicates that collagen gels modified or added with glucosaminoglycans, (e.g. 5% or 10% chondroitin sulfate or less than 5% of HA) would enhance the cell growth and adhesion, the growth and adhesion of cells would be inhibited if more than 5% HA was incorporated into collagen gels.

(7 ) S. Srivastava et al., 1990, Biomaterials, Vol., 11, pages 162-168, studied the effect of the collagen or modified collagen on the growth of fibroblast cell line. The preparation of collagen/GAGs and fibronectin composite materials were following the method described by Yannas. 3% w/v of degassed collagen slurry was stirred in 0.05M acetic acid while a solution of HA dissolved in 0.05M acetic acid was added into the resultant solution until the dry weight of GAGs was 2.5% based on the weight of collagen, and then solution was homogenized and degassed. Collagen/HA composite material contains 5%, 10%, or 20% GAGs, and collagen/CS composite material contains 5% or 10% chondroitin-4-sulfate and chondroitin-6-sulfate. Their preparation method was the same as the above described. 1% Fibronectin was further added into the above composite material, and placed on the petri dish for culturing cell. Experimental results showed that polystyrene was better than nature collagen to be a material of petri dish, but the adhesion of collagen was improved by chemical modification or by adding with fibronectin and chondroitin-4-sulfate. If content of HA was more than 5%, however, the cell adhesion and growth of nature collagen matrix could be better than the polystyrene material.

(8) M. Hanthamrongwit et al., 1996, Biomaterials, Vol., 17, pages 775-780, studies the effect of the glycosaminoglycans, hyaluronic acid and chondroitin 6-sulfate, diamines and carbodiimides cross-linking agents on the growth of human epidermal cells in collagen gels. Collagen gel (0.3% w/v) was prepared by mixing 4.2 mg/ml collagen solution, a mixture of 10 times volume ofDMEM and 0.4MNaOH (2:1),and 1:100 (v/v) acetic acid at a ratio of 7:1:2, and adjusting the solution at pH 8-8.5 by addition of 1M NaOH. The gels were stood for 2hrs at room temperature. If intend to add GAG, hyaluronic acid and chondroitin-6-sulfate solutions in serum-free DMEM were used to substitute for acetic acid used in the above solution at various ratio. After forming gels, 1-ethyl-3-(3-dimthyaminopropyl)-carbodiimide and diamine were incorporated into the gels to subject to cross-linking reaction.

(9) L. H. H. Olde Damink et al., 1996, Biomaterials, Vol., 17, pages 765-773, describes that non-crosslinked dermal sheep collagen (N-DSC) was cross-linked with 1-ethyl-3-(3-dimthyaminopropyl)-carbodiimide (EDC) to give E-DSC by immersing 1 g N-DSC samples (1.2 mmol) in 100 ml of an aqueous solution containing 1.15 g (6.0 mmol) EDC at room temperature for 18 hrs. During the reaction, a pH of the solution was maintained at 5.5 by addition of 0.1M HCl. The molar of N-DSC samples was calculated assuming that 120 carboxylic acid group residues are present per α-chain (˜1000 amino acids ) and that each α-chain has a molecular weight of 100,000. After cross-linking, E-DSC samples were rinsed for 2 hrs in a 0.1M Na₂HPO₄ solution and subsequently washed four times with distilled water before lyophilization. Alternatively, cross-linking reaction of N-DSC with EDC and N-hydroxysuccinimide (NHS) to give E/N-DSC was performed by immersing N-DSC samples in aqueous-solution containing EDC and NHS at room temperature for 4 hrs. The results showed that addition of N-hydroxylsuccinimide to the EDC-containing cross-linking solution (E/N-DSC) increased the rate of cross-linking.

(10) Yannas et al., 1997, U.S. Pat. No. 4,060,081 discloses multilayer membrane which is consisting of a first and a second layers and is useful as synthetic skin. Preferred materials for the first layer are cross-linked composites of collagen and a muco-polysaccharide. The second layer is formed from a nontoxic material which controls the moisture flux of the overall membrane.

(11) Yannas et al., 1981, U.S. Pat. No. 4,280,954 discloses a method for preparing cross-linked collagen-muco-polysaccharide composite materials. A collagen solution at pH 3.2 and muco-polysaccharide solution (weight ratio is 6%-15% by weight) were mixed together, and then a precipitate of aldehyde covalent cross-linked collagen-muco-polysaccharide composite was formed.

(12) Yannas et al., 1982, U.S. Pat. No. 4,350,629 discloses that if collagen fibrils in an aqueous acidic solution(<pH 6.0) are contacted with a cross-linking agent (glutaraldehyde) before being contacted with glycosaminoglycan, the produced materials exhibits extremely low thrombogenicity. Such materials are well suited for in-dwelling catheters, blood vessel grafis, and other devices that would keep contacting with blood for a long period.

(13) Yannas et al., 1984, U.S. Pat. No. 4,448,718 discloses a process for preparing a cross-linked collagen-glycosaminoglycan composite material which comprises forming an uncross-linked composite material from-collagen and a glycosaminoglycan and contacting the uncross-linked composite with a gaseous aldehyde until a cross-linked product having an average molecular weight of from about 800 to about 60,000 is formed.

(14) Balazs et al., 1986, U.S. Pat. No. 4,582,865 discloses a method for preparing cross-linked gels of hyaluronic acid and a product containing the gels. The cross-linking HA or HA/hydrophilic polymers (polysaccharide or protein) and divinyl sulfone was carried out in a solution at 20° C. and a pH of less than 9. In the 1%-8% dry solids content of mixture, HA comprises 5%-95% of the dry solids content.

(15) Liu et al., 1999, U.S. Pat. No. 5,866,165 discloses a matrix and a method for preparing the same, which matrix is provided to support the growth of bone or cartilage tissue. A polysaccharide is reacted with an oxidizing agent to open sugar rings on the polysaccharide to form aldehyde groups. The aldehyde groups are reacted with collagen to form covalent linkages between them. Collagen and polysaccharide used to form matrix are present in a range of 99:1 to 1:99 by weight, respectively. From 1% to 50% of the repeat units in polysaccharide are oxidized and ring-opened.

(16) Pitaru et al., 1999, U.S. Pat. No. 5,955,438 discloses a method for producing a collagen matrix which may be formed into a membrane useful in guide tissue regeneration. A collagen matrix comprises collagen fibrils are incubated with pepsin in a solvent, and are then cross-linked to one another by a reducing sugar. Finally, the matrix is subjected to critical point drying.

(17) Pierschbacher et al., 1999, U.S. Pat. No. 5,955,578 discloses a method for producing polypeptide-polymer conjugates capable of healing wound. In this reference, a synthetic polypeptide comprising an amino acid sequence of dArg-Gly-Asp is bonded to a biodegradable polymer via a glutaraldehyde cross-linking agent. The resultant conjugates are used for promoting cell attachment and migration.

(18) Hall et al., 1998, U.S. Pat. No. 5,800,811 discloses a method for producing an artificial skin. The artificial skin is prepared by impregnating a collagen in a transforming growth factor-beta and then incubating with a source of stem cells.

(19) Stone et al., 1989, U.S. Pat. No. 5,880,429 discloses a method for producing a prosthetic meniscus. A pore size in the range 10-50 microns of prosthetic meniscus is formed by type I collagen fibrils (65%-98% by dry weight ) and glycosaminoglycan molecular (clondroitin-4-sulfate ; chondroitin-6-sulfate; dermatan sulfate or hyaluronic acid; 1%-25% by dry weight) and which is adapted for in growth of meniscal fibrochondrocytes.

(20) Stone, 1992, U.S. Pat. No. 5,108,438 discloses a method for producing a prosthetic inter-vertebral disc. The disc includes a dry, porous, volume matrix of bio-compatible and bio-degradable fibers which may be interspersed with glycosaminoglycan molecules (0-25% by dry weight).

The cross-linking agent is selected from the group consisting of glutaraldehyde, carbodiimides, and so on.

(21) Silver et al., 1987, U.S. Pat. No. 4,703,108 discloses a method for preparing biodegradable collagen-based matrix in sponge or sheet form. HA and collagen are added to a diluted HCl solution of pH 3.0 and the mixture is homogenized in a blender. The solution is then poured into a vacuum flask and de-aerated under a vacuum, and then cross-linked with carbodiimide. After then, the matrix is allowed to air dry or freeze dry. The product of collagen-based matrix is cross-linked by immersion in an aqueous solution containing 1% by weight of cyanamide at pH 5.5 for a period of 24 hrs at 22° C. Hereinafter, the matrix is frozen and dried at −65° C. under a vacuum.

(22) Silver et al., 1990, U.S. Pat. No. 4,970,298 discloses a porous biodegradable collagen sponge-like matrix which enhances the healing of wound. Collagen is dispersed in an acid solution of pH from 3.0 to 4.0 and mixed with the fibronectin in an acid solution of pH 3.0 to 4.0 in a blender. Collagen dispersions to be converted into sponge are frozen at −100° C. before freeze drying at −65° C. The matrix is cross-linked in two steps consisting of first cross-linking with carbodiimide and then subjecting to dehydrothermal, or first subjecting to dehydrothermal and then cross-linking with carbodiimide.

### SUMMARY OF THE INVENTION

Based on the reports of the patents and references above-mentioned, the general preparation of the polysaccharide-protein bio-composites is under an acidic condition, a polysaccharide-protein fiber precipitate is formed by forming ionic bond between polysaccharide and protein from mixing minor amount of polysaccharide (less than 15% weight of collagen) and protein, and the resultant precipitate is further cross-linked with a cross-linking reagent to form a covalent bond, a non-directional fiber sponge or porous matrix is produced after washing, filtration and pore-formation. A defect of this process is that a non-homogeneous porous matrix having fiber structure, other than a homogenous composite, be produced, it is difficult to form impalpable matrices in a form of suitable shape as desired. If a shape is required, a piece of the prepared precipitate is generally homogenized by chopping it into small segments, and the homogenized slurry was then poured into a mold having the desired shape, then lyophilized. According to method of the present invention, a solution of polysaccharides and a solution of protein each having different pH value are prepared, mixed at various ratio, and then processed into bio-composites having the shape as desired (such as membrane, sponge, fiber, tube or micro-granular and so on). Subsequently, the bio-composites is subjected to a cross-linking reaction in a mixture of water and organic solvent to obtain impalpable bio-composites which is a homogeneous, bio-compatible, biodegradable, and has excellent physical properties and a prolonged enzymatic degradation time.

The advantage of this invention is that a homogenous polysaccharide-protein solution can be prepared under a wide range of pH value, not only under an acidic condition, and the weight ratio of polysaccharide to protein is from 2/98 to 90/10. In traditional methods, the collagen is usually used as a major component and the polysaccharide is used as an additive, the maximal ratio of polysaccharide to collagen is around 20%. Besides, the matrix solution produced from the present invention possesses a uniform density and porosity, and can be manufactured into various shapes, including membrane, sponge, fiber, tube or fine particles and so on. It can also avoid the loss of polysaccharide and reduce reaction time to only 2-4 hrs if a cross-linking reaction with carbodiimide is conducted in the presence of weak acid in organic solvents.

In prior arts, it usually uses aldehydes as a cross-linking reagent. If carbodiimide is used as a cross-linking agent, the cross-linking reaction is always conducted in water and its reaction will take place for more than 24 hrs.

There are many advantages in this invention. The techniques of the present invention have never been described in previous references. According to the method of the present invention, bio-composites which can be produced in various shapes are suitable for using in a variety of fields, including biomedical, materials engineering, histological engineering, medical equipment, pharmacy and cosmetic fields.

The present invention also relates to the use of the bio-composite in the prevention or reduction of post-surgical adhesion and in of bone regeneration.

Other features and advantages of the present invention will be apparent from the following description of the preferred embodiments thereof and the appended claims.

### BRIEF DESCRIPTION OF THE DRAWINGS

FIG. 1 shows photographs of hyaluronic acid (HA)-collagen membrane composites prior to 1-ethyl-3-(3-dimethylaminopropyl) carbodiimide hydrochloride-induced crosslinking: (A) HA:collagen=60:40 (w/w); (B) HA:collagen=50:50 (w/w); and (C) HA:collagen=40:60 (w/w).

FIG. 2 shows photographs of hyaluronic acid (HA)-collagen membrane composites after 1-ethyl-3-(3-dimethylaminopropyl) carbodiimide hydrochloride-induced cross-linking: (A) HA: collagen=80:20 (w/w); and (B) HA:collagen=50:50 (w/w).

FIG. 3 shows the concentration of hyaluronic acid released from pieces (1×1 cm) of hyaluronic acid (HA)-collagen membrane with different compositions following incubation with hyaluronidase (activity: 200 units/ml) for 24 h at 37° C. (mean±SD). The amount of in vitro enzyme digestion was determined by measuring the HA concentration of the solution using the carbazole reagent.

FIG. 4 shows photomicrographs of L929 fibroblasts taken under phase contrast after culture for 24 h on (A) a polystyrene Petri dish and (B) a hyaluronic acid-collagen (50/50, w/w) membrane cross-linked with 1-ethyl-3-(3 -dimethylaminopropyl) carbodiimide hydrochloride. The fibroblasts have been stained with neutral red to indicate cell viability. ×40.

FIG. 5 shows there was no adhesion between the peritoneal defects covered with hyaluronic acid-collagen membranes and the intraperitoneal visceral organs at week 1 post-surgery.

FIG. 6 shows a photograph of the peritoneal defects covered by material C (hyaluronic acid: collagen=40:60 [w/w]) showing 20-80% adhesion at week 4 post-surgery; arrows indicate the adhesions.

FIG. 7 shows the mononuclear cell reaction associated with the use of material D that was only observed at 1 week after surgery (haematoxylin and eosin; ×100).

FIGS. 8(A) and (B) show the axial magnetic resonance image between the dura and the surrounding scar tissues 3 months after laminectomy. With membrane B-treated, a hyposignal space (arrow) was seen at the surgery site (A) when compared to a continuity (arrow) in the control (B).

FIGS. 9(A-C) show sagittal magnetic resonance images. Less scarring (S) at the Membrane A—(A) and Membrane B—(B) treated laminectomy sites when compared to dense scar formation in the control (C). The scar tissues abutted on the spinal canal (C) in the control.

FIG. 10(A) shows an identifiable plane between the regenerated bone (B) and the dura (arrow) at Membrane B-treated laminectomy site, and the scar tissues (S) above the regenerated bone are scanty and replaced by fibrofatty tissues. FIG. (B) shows the defect site of the control demonstrating severe to moderate peridural scar adhesion between the regenerated bone (B) and the dura (arrow) with moderate amount of scar tissue (S). (H & E, original magnification 12.5×).

FIG. 11 shows displacement of the membrane (arrow), exposing a space for scar tissue (S) to come close to the peridural space. (H & E, original magnification 12.5×).

FIG. 12 shows statistical graph of the effect of different time intervals on amount of scar formation of each group.

FIG. 13 shows statistical graph of the effect of HA/collagen membranes and control on amount of scar formation of different time periods.

### DETAILED DESCRIPTION OF THE PREFERRED EMBODIMENTS

The present invention relates to a method for producing polysaccharide-protein bio-composites in any desired shapes. The advantage of the method is that the bio-composite can be produced into various shapes, such as membrane, sponge, fiber, tube, and micro-granular. After further subjecting to cross-linking reaction, a bio-composite is formed, which is bio-compatible, biodegradable, non-toxic, and impalpable, and possesses prolonged enzymatic degradation time and excellent mechanical strength. It is extremely suitable for the application in biomedicine, histological engineering, materials engineering, medical equipment and cosmetic fields. The bio-composite prepared by the present method is suitably used as hemosats, vascular sealants, orthopedic implant coatings, vascular implant coatings, dental implants, wound dressings, anti-adhesion barriers, platelet analyzer reagents, research reagents, engineering of cartilage, artificial tendons, blood vessels, nerve regeneration, cornea implants, cell preserving solutions and for delivering growth factor and/or drugs. According to the present invention, the prepared bio-composite can be further processed into various products possessing high additional value. It is very useful for commercial utilization.

The present invention relates to a method for producing polysaccharide-protein bio-composites, comprising the steps of:

(a) preparing a mixture of a polysaccharide and a protein in a solution at a weight ratio of polysaccharide to protein in a range of 20/80 to 80/20;

(b) adjusting the mixture at a pH value between 3 and 11 by adding either an acid or a hydroxide, forming into a matrix having a desired shape, such as membrane, porosity, sponge, tube or micro-granular and so on;

(c)—subjecting the matrix to cross-linking reaction by using a cross-linking agent in a mixture of water and one or more organic solvents

In the method of the present invention, the crosslinking reaction is preferably performed in a mixture of water and organic solvents containing across-linking reagent under a pH of from 4 to 4.5 at a temperature of from 20 to 45° C. for a period of 1 to 6 hours, preferably 2 to 4 hours;

The method of the present invention further comprises the step of:

(d) washing the matrix for several times, and immersing in a salt aqueous solution which is selected from the group consisting of sodium chloride, dibasic sodium phosphate, or a mixture thereof.

The matrix was then further washed several times with large volumes of de-ionized water and pore-formation; As to the manner of the pore-formation, it includes, but not limited to, (1) freeze-drying method, (2) supercritical CO₂ foaming method, (3) phase immersing method, (4) critical point drying method, (5) fiber meshes method, (6) membrane, lamination, and (7) particulates leaching method.

In the Step (a), the polysaccharide is chosen from the group consisting of hyaluronic acid, carboxymethyl cellulose, pectin, starch, chondroitin-4-sulfate, chondroitin-6-sulfate, alginate, chitosan, agar, carragenan and guar gum, and a mixture thereof.

In the step (a), the protein is chosen from the group consisting of collagen, gelatin, or a mixture thereof.

In the step (b), the preferred pH value is in a range between 3 and 11, and if an intended pH is less than 7, it is adjusted by adding acetic acid, hydrochloric acid, or a mixture thereof. If an intended pH is more than 7, it is adjusted by adding sodium hydroxide, potassium hydroxide, or a mixture thereof.

The solids content of resultant polysaccharide-protein mixture is in a range between 0.2% and 4.0% by weight, and the percent of polysaccharide is in a range between 2% and 98%, based on the total weight of the mixture.

As to the procedures for forming the matrix into different shapes in the step (c) are illustrated in details as follows:

(1) The matrix is prepared as a film matrix by casting the degassed matrix consisting of polysaccharide and protein solutions into a mold and drying in an oven at a temperature of 35° C. to yield a film matrix.

(2) The matrix is prepared as a porous matrix by casting the degassed matrix consisting of polysaccharide and protein solutions into a mold in a refrigerator at a temperature of −80° C. and drying at a vacuum to yield a porous matrix having a inter-connective porous structure.

(3) The matrix is prepared as a powder matrix by dropping the degassed matrix consisting of polysaccharide and protein solutions into the freezing solution at a temperature of −80° C. by using a syringe, and pore-forming to yield powder matrix. Such a pore-forming procedure can be carried through the following manner: (1) freeze-drying method, (2) supercritical CO₂ foaming method, (3) phase immersing method, (4) critical point drying method, and (7) particulates leaching method.

(4) The matrix is prepared as a fiber matrix by squeezing the degassed matrix consisting of polysaccharide and protein solutions into a coagulant solution in a mixture of water and organic solvents, and pore-forming to yield a fibrous matrix having a thickness of from 50 um to 1 mm. Such a pore-forming procedure can be carried through the following manner: (1) freeze-drying method, (2) supercritical CO₂ foaming method, (3) phase immersing method, (4) critical point drying method, (5) fiber meshes method, (6) membrane lamination, and (7) particulates leaching method.

The organic solvent contained in the coagulant solution is chosen from the group consisting of 1,4-dioxane, chloroform, methylene chloride, N,N-dimethylformamide, N,N-dimethylacetamide, ethyl acetate, acetone, methyl ethyl ketone, methanol, ethanol, propanol, isopropanol, butanol, and a mixture thereof; the percentage of the organic solvent in the coagulant solution is between 60% and 100% by weight, preferably between 75% and 100% by weight. The preferred organic solvent is a mixture of ketones and alcohols.

The cross-linking agent in step (d) is preferably a carbodiimide, which is selected from the group consisting of 1-methyl-3-(3-dimethylaminopropyl)-carbodiimide, 3-(3-dimethy laminopropyl)-3-ethyl-carbodiimide, 1-ethyl-3-(3-dimethylaminopropyl)-carbodiimide or any mixture thereof.

The mixture of water and organic solution in the method of the present invention is preferably consisting of 5%-50% by weight of water and 95-50% by weight of either ethanol or acetone, or the both, preferably consisting of 5%-30% by weight of water and 95-70% by weight of either ethanol or acetone, or the both.

The salt aqueous solution in step (d) is used at a concentration of 0.15-4M. The immersion time is in a range between 30mins and 3 hrs.

The present invention is described in more detail in the following example. These examples are giving by way of illustration and are not intended to limit the invention except as set forth in the claims.

### EXAMPLE 1A-1G

### Preparation of Hyaluronic Acid/Collagen Matrix

Hyaluronic acid (HA)(60 mg) and collagen(40 mg) were each dissolved in different solvent as shown in table 1, and then the prepared two solutions were mixed together to form a mixture that a weight ratio of HA to collagen is 3 to 2 and a solid content of the mixture is 1%.

The resulting mixture was cast into a mold made of Teflon to yield a film. The films prepared in Example 1D and 1E had the optimal morphology and physic properties.

TABLE 1

| | Example | | | | | | |
|---|---|---|---|---|---|---|---|
| | 1A | 1B | 1C | 1D | 1E | 1F | 1G |
| HA^{a} | H₂O | 0.1N | 0.1M | H₂O | H₂O | H₂O | H₂O |
| solvent | | NaCl | CH₃COOH | | | | |
| Collagen | 0.5M | 0.1M | 0.1N | 0.1M | Dissolving in | Dissolving | A mixture |
| | CH₃COOH | CH₃COOH | NaCl | CH₃COOH | 0.5M acetic | in water | of 0.5M |
| | | | | | acid, | and then | CH₃COOH |
| | | | | | Then | adjusting | and 1N |
| | | | | | adjusting pH | pH 7 by | NaOH |
| | | | | | by 1N NaOH | HCl | |
| NaCl | — | — | — | 30 mg | — | — | — |
| mixed | white fiber | transparence | transparence | transparence | transparence | fine fiber | white fiber |
| solution | precipitate | and low | | | | precipitate | precipitate |
| | | viscosity | | | | | |
| 1N | few drops, | — | — | — | — | — | — |
| NaCl | fiber | | | | | | |
| | precipitate | | | | | | |
| | and then | | | | | | |
| | dissolved | | | | | | |
| PH | ˜9 | ˜8 | ˜7 | ˜3 | ˜6 | ˜7 | ˜6 |
| morphology | fine fiber | semi | semi | white and | White, | fine fiber | white |
| | on the | transparence | transparence | dense | dense and | on the | |
| | matrix | | | | high | matrix | |
| | surface | | | | toughness | surface | |

### EXAMPLE 2

### Preparation of HA/Gelatin Matrix

HA (50 mg) was dissolved in 5ml of pure water. Separately, gelatin (50 mg) was dissolved in 5 ml of warm water(more than 55° C.) and then added with sodium chloride (30 mg).

The prepared two solutions were mixed together to form a 10 ml mixture of which pH is around 6.5, the weight ratio of HA to collagen is 1 to 1 and a solid content is 1%.

The resulting solution was cast into a mold made of Teflon and allowed to dry in an oven to yield a transparent film.

### EXAMPLE 3

### Preparation of HA/Collagen Matrix at Different Salt Concentration after Neutralization

HA (60 mg) was dissolved in pure water. Separately, collagen (40 mg) was dissolved in 0.5M acetic acid solution, and then neutralized with sodium hydroxide. Adjust the salt concentration after neutralization and maintain the pH at 6 by adding various volume of water, acetic acid and sodium hydroxide as shown in Table 2. The prepared two solutions were mixed together to form a 10 ml mixture in which a weight ratio of HA to collagen is 3 to 2 and a solid content is 1%.

The resulting solution was cast into a mold made of Teflon and allowed to dry in an oven to yield a film.

TABLE 2

| | Example | | |
|---|---|---|---|
| | 3A | 3B | 3C |
| H₂O (ml) | 5.5 | 7.0 | 8.5 |
| 0.5M CH₃COOH | 3.0 | 2.0 | 1.0 |
| 1N NaCl | 1.5 | 1.0 | 0.5 |
| Salt conc of | 0.15 | 0.1 | 0.05 |
| neutralization. (M) | | | |

### EXAMPLE 4

### Preparation of HA/Collagen Matrix at Different pH

HA(60 mg)was dissolved in pure water. Separately, collagen(40 mg) was dissolved in 0.5M acetic acid solution, and then neutralized with sodium hydroxide. Adjust a pH value by adding various volumes of acetic acid and sodium hydroxide as shown in Table 3 and maintain a salt concentration after neutralization at 0.15M. The prepared two solutions were mixed together to form a 10 ml mixture in which a weight ratio of HA to collagen is 3 to 2 and a solid content is 1%.

The resulting solution was cast into a mold made of Teflon and allowed to dry in an oven to yield a transparent film.

TABLE 3

| | Example | | |
|---|---|---|---|
| | 4A | 4B | 4C |
| H₂O (ml) | 3.5 | 5.5 | 5.44 |
| 0.5M CH₃COOH | 5.0 | 3.0 | 3.0 |
| 1N NaCl (ml) | 1.5 | 1.5 | 1.56 |
| PH value | 4.7 | 6.0 | 11.0 |

### EXAMPLE 5

### Preparation of HA/Collagen Matrix at Different Ratio

HA was dissolved in pure water. Separately, collagen was dissolved in 0.5M acetic acid solution, and then neutralized with sodium hydroxide. A salt concentration after neutralization is maintained at 0.15M, a pH is maintained at 4.7, and the volume ratio of added water, acetic acid and sodium hydroxide is maintained at 3.5:5:1.5. The prepared two solutions were mixed together to form a I Oml mixture in which a weight ratio of HA to collagen is as shown in Table 4 and a solid content is 1%.

The resulting solution was cast into a mold made of Teflon and allowed to dry in an oven to yield a transparent film.

TABLE 4

Example

| | 5A | 5B | 5C | 5D | 5E | 5F |
|---|---|---|---|---|---|---|
| HA (mg) | 90 | 80 | 60 | 50 | 20 | 2 |
| Collagen (mg) | 10 | 20 | 40 | 50 | 80 | 98 |
| Weight ratio | 9:1 | 4:1 | 3:2 | 1:1 | 1:4 | 1:49 |
| (HA/collagen) | | | | | | |

### EXAMPLE 6

### Preparation of HA/Collagen Matrix at Different Solid Content

HA was dissolved in pure water. Separately, collagen was dissolved in 0.5M acetic acid solution, and then neutralized with sodium hydroxide. Maintain a salt concentration after neutralization at 0.15 M and a pH at 4.7, the volume ratio of added water, acetic acid and sodium hydroxide is at 3.5:5:1.5. The prepared two solutions were mixed together to form a 10 ml mixture in which a weight ratio of HA to collagen is 3 to 2 and a solid content is as shown in Table 5.

The resulting solution was cast into a mold made of Teflon and allowed to dry under oven to yield a transparent film.

TABLE 5

| | Example | | |
|---|---|---|---|
| | 6A | 6B | 6C |
| HA (mg) | 120 | 60 | 30 |
| Collagen (mg) | 80 | 40 | 20 |
| Solid content (%) | 2 | 1 | 0.5 |

### EXAMPLE 7

### Preparation of HA/Collagen Matrix in a Fiber Form

HA (100 mg) was dissolved in 3.5 ml of pure water. Separately, collagen (100 mg) was dissolved in 5 ml of 0.5M acetic acid solution, and then neutralized with 1.5 ml of 1N sodium hydroxide. The salt concentration of neutralization is 0.15M. The prepared. two solutions were mixed together to form a mixture in which a pH of solution is around 4.7, a weight ratio of HA to collagen is 1 to 1 and a solid content is 2%.

The resulting solution was continuously pressed into a 95% alcohol solution to form a mono-filament fiber by using syringes having various sizes, and allowed to dry in an oven to yield a HA-protein matrix.

### EXAMPLE 8

### Preparation of HA/Collagen Matrix in a Form of Micro-Granular

HA (100 mg) was dissolved in 3.5 ml of pure water. Separately, collagen. (100 mg) was dissolved in 5 ml of 0.5M acetic acid solution, and then neutralized with 1.5 ml of 1N sodium hydroxide. The salt concentration after neutralization is 0.15M. The prepared two solutions were mixed together to form a mixture in which a pH of the mixture is around 4.7, a weight ratio of HA to collagen is 1 to 1 and a solid content is 2%.

The micro-granular matrix was formed by dropping the resulting mixture into the liquid nitrogen and then pore-forming. The pore-forming can be achieved by freeze-drying, supercritical CO₂ foaming, phase immersing, critical point drying, and particulates,leaching methods.

### EXAMPLE 9

### Preparation of HA/Collagen Matrix in a Porous Form

HA (100 mg) was dissolved in 3.5 ml of pure water. Separately, collagen (100 mg) was dissolved in 5 ml of 0.5M acetic acid solution, and then neutralized with 1.5 ml of 1N sodium hydroxide. The salt concentration after neutralization is 0.15M. The prepared two solutions were mixed together to form a mixture in which a pH of solution is around 4.7, a weight ratio of HA to collagen is 1 to 1 and a solid content is 2%.

The resulting solution was cast into a mold made of Teflon at a temperature of −80° C. and allowed to dry to yield a porous sponge matrix after pore-formation. The pore-formation can be achieved by freeze-drying, supercritical CO₂ foaming, phase immersing, critical point drying, fiber meshes, membrane lamination, and particulates leaching methods.

### EXAMPLE 10

### The Effect of Cross-Linked Agent on Cross-Linking Reaction of HA/Collagen Matrix

The film of Example 6A was chopped to pieces in equal size and immersed in the EDC to subject to cross-linking reaction at 30° C. for 2 hours (experimental conditions were shown in Table 6). The mixture was then washed 3 times with 80% acetone aqueous solution, each washing time is 20 mins. After then, the mixture was further washed 3 times with de-ionized water, each washing time is also 20 mins. Finally, the mixture was spread on a substrate and dried. The cross-linked film was subject to swelling test by immersing in 0.15M sodium chloride solution, incubating for 5 days with gentle shaking at 37° C., then the swelling behavior was observed. From the results shown in Table 6, it showed that in order to avoid the dissolution of matrix and enhance the cross-linking efficiency, the cross-linking of matrix was only carried out in a mixture of water and organic solvent (Examples 10D, 10E)

TABLE 6

| | Example | | | | |
|---|---|---|---|---|---|
| | 10A | 10B | 10C | 10D | 10E |
| EDC conc. | 2.3 | 2.3 | 2.3 | 2.3 | 2.3 |
| (wt %) | | | | | |
| Solvent | H₂O | PH 4.7 | PH 4.8 | 80% | 80% |
| | | solution | solution | ethanol | acetone |
| Morphology | thinness | thinness | thinness | normal | normal |
| Dissolving test | Soluble | soluble | soluble | insoluble | insoluble |

### EXAMPLE 11

### The Effect of a Concentration of Cross-Linked Agent on the Cross-Linking Reaction of HA/Collagen Matrix

The film of Example 6A was chopped to pieces in equal size and immersed in 80% acetone solution containing EDC at pH 4.7 and at 30° C. for 2 hrs (experimental conditions were shown in Table 7) The mixture was then washed 3 times with 80% acetone solution, each washing time is 20 mins. After then, the mixture was further washed 3 times with de-ionized water, each washing time is also 20 mins. Finally, the mixture was spread on a substrate and dried. The cross-linked film was subjected to swelling test by immersing in 0.15M sodium chloride solution, incubating for 5 days with gentle shaking at 37° C., then the swelling behavior was observed. Hyaluronidase (220 U/ml ) was dissolved in 0.15M sodium chloride. Film was weighted and put into the enzyme solution for testing enzyme degradability of the film. After 24 hours, the solution was taken out for uronic acid assay, and then the percent of hydrolysis of HA film was calculated. From the results in Table 7, it showed that the rate of enzyme degradation of the cross-linked film prepared by the present method was reduced significantly.

TABLE 7

| | Example | | | | |
|---|---|---|---|---|---|
| | 11A | 11B | 11C | 11D | Control |
| EDC (wt %) | 0.625 | 1.25 | 2.5 | 5 | — |
| Dissolving test | insoluble | insoluble | insoluble | insoluble | soluble |
| HA enzyme | 1.87 | 1.5 | 0.68 | 1.02 | 31.13 |
| degradation | | | | | |
| (%) | | | | | |

### EXAMPLE 12

### Cross-Linking Reaction of a Porous HA/Collagen Sponge Matrix

The porous sponge of Example 9 was placed in an oven at 110° C. and under a vacuum for 3 hrs. The dried specimens were then immersed in a 80% acetone solution for 30 mins, and then transferred to a 80% acetone solution containing 2.5% EDC at pH 4.7.

The specimens were taken out after reaction at 30° C. for 2 hours, and then washed 3 times with 80% acetone, each washing time is 20 mins. After then, the specimens were further immersed in 1M sodium chloride for 20 mins, and washed 3 times with de-ionized water, each washing time is also 20 mins. Finally, the specimens were spread on a substrate and dried.

### EXAMPLE 13

### Determination of Ability of Growth of Cell and Cyto-Toxicity of Cross-Linked HA/Collagen

The films prepared from Examples 5C, 5D and 5E were immersed in the 80% acetone solution containing 2.5% EDC at pH 4.7. The film was taken out after reaction at 30° C. for 2 hours, and then washed 3 times with 80% acetone, each washing time is 20 mins. After then, the film was further immersed in 1M sodium chloride for 20 mins, and washed 3 times with de-ionized water, each washing time is also 20 mins. Finally, the film was spread on a substrate and dried.

The cross-linked film matrix was placed in a cell culture plate. Immortalized mouse 3T3 fibroblast cell and human fibroblast cell were seeded on the film matrix for observing the growth of cell (Please refer to Tables 8,9). The results of cell seeding experiment showed that cell can growth well on the film matrix, and all the cells were alive. Also, it is stained with neutral red dye and showed that the film matrix was non-toxic to the human and mouse cell growth.

TABLE 8

| | Seeding of | 1st day | 2nd day | Third day |
|---|---|---|---|---|
| | cell No | (×10⁴ | (×10⁴ | (×10⁴ |
| Example | (×10⁴ cell/ml) | cell/ml) | cell/ml) | cell/ml) |
| Cross-linked 5C | 4 | 1.8 | 2.4 | 4.8 |
| Cross-linked 5D | 4 | 2.4 | 4.2 | 7.4 |
| Cross-linked 5E | 4 | 1.4 | 1.8 | 3.4 |

TABLE 9

| | | 1st day | 2nd day | Third day |
|---|---|---|---|---|
| | Seeding of cell | (×10⁴ | (×10⁴ | (×10⁴ |
| Example | No (×10⁴ cell/ml) | cell/ml) | cell/ml) | cell/ml) |
| Cross-linked 5C | 4 | 1.2 | 2.2 | 5.0 |
| Cross-linked 5D | 4 | 2.6 | 4.4 | 7.4 |
| Cross-linked 5E | 4 | 1.6 | 2.4 | 4.0 |

The present inventors aimed to develop a biocompatible 1-ethyl-3-(3-dimethylaminopropyl) carbodiimide hydrochloride (EDC)-cross-linked HA-collagen composite and to evaluate its effect on post-surgical intraperitoneal adhesion in rats.

The effects of the bio-composite according to the present invention on post-surgical adhesion will be further illustrated by reference to the following non-limited experimental examples.

Experimental Section

### EXPERIMENTAL EXAMPLE 1

Materials and Methods

Materials

Collagen (type I/III=85/15) was purchased from Meddicoll (Sydney, Australia). The sodium salt of hyaluronic acid (molecular weight: 1.5×10⁶) was purchased from LifeCore (MN, USA) as a dry powder. EDC and testicular hyaluronidase were purchased from Sigma (St Louis, Mo., USA). The commercially produced anti-adhesion film used in this study, Seprafilm® (Genzyme Corporation, Cambridge, Mass., USA) is a biopolymer composed of sodium HA and carboxymethylcellulose (CMC). All other chemicals were of chemical grade.

Preparation of Cross-Linked HA-Collagen Membranes

Collagen was dissolved in 0.5 M acetic acid at 4° C. and 1N NaOH was used to adjust the pH to 5.0. The collagen solution was mixed with the HA aqueous solution to produce a range of weight ratios (HA: collagen) from 80/20 to 40/60 (w/w). An aliquot (10 ml) of the HA-collagen solution was poured into a 5×5 cm Teflon dish and allowed to dry at 35° C. to form a fabricated membrane.

To cross-link the fabricated HA-collagen membranes, they were immersed in 2.5% EDC solution in 80% acetone for 2 h at 35° C. The HA-collagen membranes were then removed from the Teflon plate, washed three times with 80% acetone for 30 min each to remove the residual EDC and washed again with distilled water. The washed HA-collagen membranes were allowed to dry under atmospheric pressure at 25° C.

In Vitro Enzyme Degradation

An in vitro enzyme degradation test of the cross-linked HA-collagen membranes was performed using hyaluronidase buffer, which had an activity of 200 units/ml. Pieces (1×1 cm) of dried cross-linked HA-collagen membranes were immersed in hyaluronidase buffer at 37° C. for 24 h. The amount of in vitro enzyme digestion was determined by measuring the HA concentration of the solution after 24 h using the carbazole reagent. Non-cross-linked HA-collagen membranes were used as control membranes.

Cytotoxicity Test

The cytotoxicity of EDC-treated HA-collagen membranes was tested by a direct contact cell culture method using L929 fibroblasts (CCRC 60091, NCTC clone 929, from mouse connective tissue) and by staining the lysosomes of living cells with neutral red (3-amino-7-dimethylamino-2-methyphenazine hydrochloride). L929 fibroblasts were cultured in Eagle's MEM (GIBCO™, Invitrogen Corp, Calif., USA) with non-essential amino acids containing 90% Earle's BSS, 10% horse serum and 10% fetal bovine serum (FBS, Hyclone Laboratories Inc., Utah, USA) at 37° C. in an atmosphere of 5% CO2. The HA-collagen membranes were shaped to fit the wells of a 24-well plate and placed into the wells under a glass ring (to hold them at the bottom of the well). L929 fibroblasts were sub-cultured into the 24-well plates at a density of 8×10⁴/ml³. After 24 h incubation, the tissue culture medium was removed and the cells stained with 0.005% neutral red solution for 3 h to identify the living cells. A polystyrene Petri dish was used to generate control L929 cells, which adhered to and spread normally on the plastic surface of the dish.

Cells were examined after staining using a NIKON ECLIPSE TS100 light microscope and photomicrographs taken with a SONYSSC-DC50A digital system.

Evaluation of INtraperitoneal Anti-Adhesion Performance

The animal studies were carried out according to the guidelines of Chang Gung Memorial Hospital's Institutional Animal Care and Use Committee. The Committee acknowledged that our animal studies complied with the law protecting animals issued by the Council of Agriculture Executive Yuan, Republic of China and guidelines cited in the Guide for the Care and Use of Laboratory Animals (Institute of Laboratory Animal Resources, National Research Council, USA).

Twenty-four Sprague-Dawley rats were fasted overnight. Anaesthesia was administered by an intraperitoneal injection of ketamine (100 mg/kg) and xylazine (20 mg/kg). The surgical technique, which was identical to that used for a clinical laparotomy, was performed under strict aseptic conditions and meticulous haemostasis was maintained. A midline incision was performed and the peritoneal cavity was exposed. Two parietal peritoneal defects (each 2×0.5 cm in size) were made on each side of the anterior abdominal wall 2 cm away from the midline. The upper and lower edges of each defect were marked with 4-0 Nylon stitches.

Four different compositions of HA-collagen membranes were tested (Table 10). The test membranes were cut into 2×1 cm strips to cover the peritoneal defects. Each animal was randomly assigned to receive two different test HA-collagen membranes. The 24 rats were randomly divided into four groups of six rats; one group was killed at 1, 2, 3 and 4 weeks after surgery.

At post-mortem, the peritoneal cavity was opened through bilateral subcostal incisions. Any adhesions that had developed between the traumatized peritoneal defects and the intraperitoneal visceral organs were evaluated by gross quantitative assessment of the adhesion(s) (percentage of area of the traumatized peritoneal defect between the two stitch-marks that had adhesions) and histological examination.

The area covered by the HA-collagen membrane was resected en bloc, fixed in 10% buffered formalin and embedded in paraffin. Sections were cut (5 μm) and stained with haematoxylin and eosin. Histological analyses were performed, using an Olympus BX51 light microscope, paying special attention to the presence of any residual HA-collagen membrane, granulocytes, macrophages or histiocytes, giant cell reactions and any fibrosis.

Results

Physical Properties of HA-Collagen Membranes

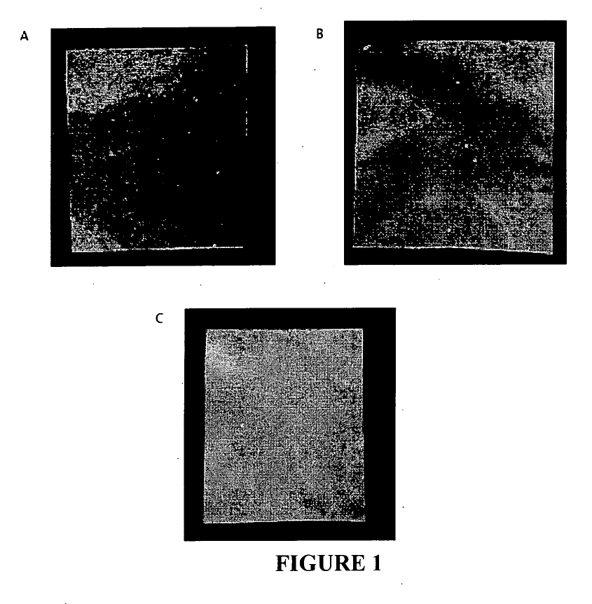

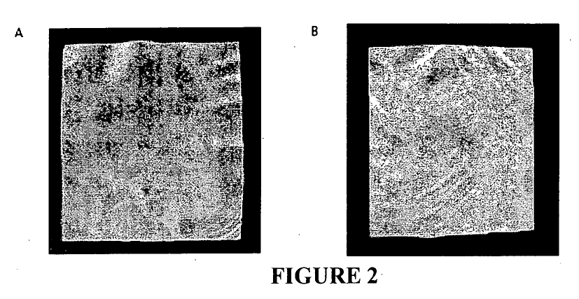

The transparency of the HA-collagen membranes varied with the weight ratio of HA to collagen. Prior to cross-linking, the membrane generally became more opaque with increasing collagen content (FIG. 1). Following ECD-induced cross-linking, all of the HA-collagen membranes, regardless of their collagen content, became opaque (FIG. 2).

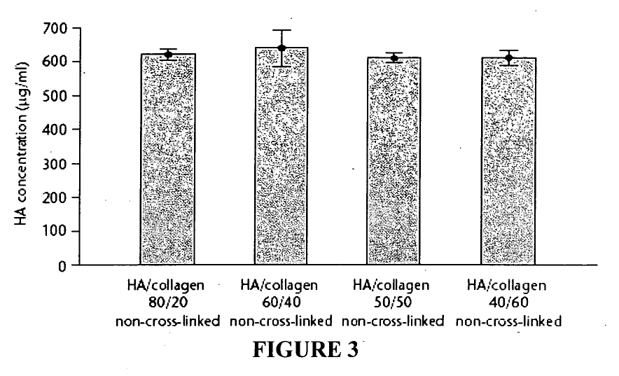

In addition to changes in appearance, the HA-collagen membranes also showed different degrees of resistance to hyaluronidase activity. Cross-linked HA-collagen membranes were more resistant to hyaluronidase activity than non-cross-linked membranes, which were readily degraded by the enzyme (FIG. 3). The amount of HA that was released into solution after treatment of non-cross-linked HA-collagen membranes with hyaluronidase for 24 h was not dependent upon the composition of the membranes (FIG. 3). Treatment of all of the various compositions of cross-linked HA-collagen membranes with hyaluronidase for 24 h did not yield significant amounts of HA (data not shown).

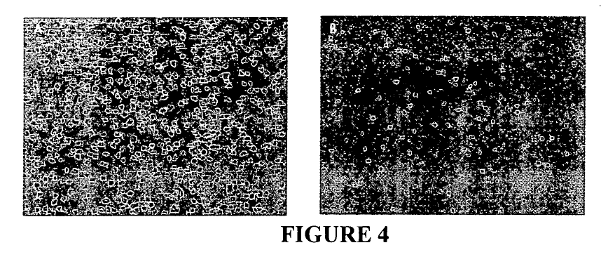

When L929 fibroblasts were cultured on HA-collagen membranes for 24 h, the cells presented with a different morphology (FIG. 4). FIG. 4A shows the normal morphology associated with the attachment and spreading of L929 fibroblasts on the plastic of the Petri dish. In contrast, when L929 were cultured on HA-collagen membrane they did not attach to the surface of the membrane and maintained a round shape without spreading (FIG. 4B). Staining with neutral red confirmed that almost every cell was alive despite not attaching to the HA-collagen membrane.

Evaulation of Intraperitoneal Anti-Adhesion Performance

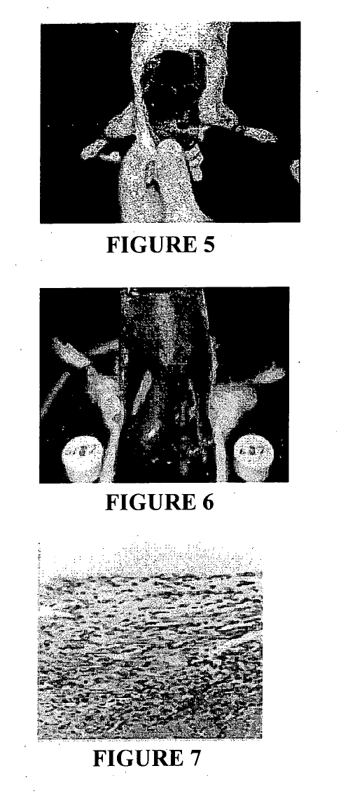

One week after surgery, gross assessment of adhesion showed that there was no adhesion between the peritoneal defects covered by all four-test HA-collagen membranes and the visceral organs (FIG. 5). Materials A, B and C remained unabsorbed, while material D was completely resorbed. At 4 weeks after surgery, material B remained unabsorbed and had migrated to distant locations. Material C had disappeared completely. Covering the peritoneal defects with material A was associated with 0-10% adhesion; covering with material B was associated with 20-70% adhesion; covering with material C was associated with 20-80% adhesion (FIG. 6); and covering with material D was associated with 20-80% adhesion. Histological examination showed that covering the peritoneal defects with material A induced mononuclear cell infiltration after weeks 1 and 2 post-surgery, but there was only mild fibrotic change at weeks 3 and 4 post-surgery. Covering the peritoneal defects with material C resulted in a severe mononuclear cell reaction at all weeks post-surgery. The use of material D was associated with a mononuclear cell reaction at week 1 post-surgery only (FIG. 7).

TABLE 10
Composition of the four hyaluronic acid (HA)-collagen membranes
used to evaluate the prevention of post-operative intraperitoneal
adhesions between surgically induced peritoneal defects and
intraperitoneal visceral organs in rats

| Material identification | Composition |
|---|---|
| Material A | HA:collagen = 60:40 cross-linked by EDC |
| Material B | HA:collagen = 50:50 cross-linked by EDC |
| Material C | HA:collagen = 40:60 cross-linked by EDC |
| Material D | Commercial product: HA-CMC composite |

EDC, 1-ethyl-3-(3-dimethylaminopropyl) carbodiimide hydrochloride;
CMC, carboxymethylcellulose

Discussion

Prior to EDC-induced cross-linking, the HA-collagen membranes exhibited various degrees of transparency depending on the amount of collagen in the composition. HA is a polymer that can absorb and retain high levels of water. When it is in solution, HA occupies a volume that is about 1000 times greater than when it is in a dry state. The HA-collagen membranes therefore became less transparent as the HA content decreased and the collagen content increased. After EDC-induced cross-linking, all of the HA-collagen membranes became opaque. The increase in opacity was likely to have resulted from the formation of cross-links between the carboxyl groups of HA with amino groups of collagen, and hydroxyl groups of collagen or HA. In addition, covalent bonding increased the resistance of the membranes to enzymatic degradation. There was no notable degradation after incubation of the cross-linked HA-collagen membranes for 24 h in a hyaluronidase solution that was 100-fold more concentrated than in vivo concentrations. When the same enzymatic degradation test was performed with the non-cross-linked HA-collagen membranes, all membrane samples were degraded extensively to the same degree. This result showed that collagen molecules did not interfere with the interaction between HA and hyaluronidase. We concluded that there was no phase separation in the microstructure of the HA-collagen membranes and that this material can last for up to at least two weeks in vivo.

Hyaluronic acid-derived material not only acts as a physical barrier, but it also acts as a biological barrier by inhibiting cell aggregation via the HA receptor. As shown in FIG. 4, L929 fibroblasts did not attach to or spread on the surface of HA-collagen membranes and maintained a round shape due to inhibition of cell adherence by HA. The L929 fibroblasts did not adhere to the HA-collagen membranes, but almost all of the cells were alive as demonstrated by their positive staining with neutral red dye. This finding indicated that there was no cytotoxicity associated with the cross-linked HA-collagen membranes following a sequential fabrication process. As a result of these biophysical properties, we would suggest that the HA-collagen membrane composite could be a candidate material for preventing post-operative adhesion.

To evaluate the anti-adhesive properties of the HA-collagen membrane composites, we chose four different composites to test in rats. Material B remains in the tissue for more than 4 weeks, cannot be easily fixed to the tissue, and may migrate to distant locations causing unnecessary tissue reactions: it is not recommended for preventing postoperative intraperitoneal adhesion. Materials A and C were readily bioresorbed within 4 weeks, showed comparable effects on adhesion prevention to the commercial product (material D), and may provide a mechanical barrier for preventing postoperative intraperitoneal adhesion.

There are several barriers currently available for clinical use, including oxidized regenerated cellulose (Interceed®; Johnson & Johnson Patient Care, Somerville, N.J., USA), hyaluronic acid-carboxymethylcellulose (Seprafilm®; Genzyme) and polytetrafluorethylene® (PTFE, Gore-Tex; Gore & Associates, Flagstaff, Ariz., USA). They still present various handling problems, however, such as being too brittle and nonbioresorptive. Taken together, our results would suggest that the HA-collagen membrane composite with a HA/collagen ratio of 60/40 may overcome some of the disadvantages of the currently available products. It was tough, was readily bioresorbed and effectively prevented postoperative intraperitoneal adhesion in a rat model.

We conclude that EDC-cross-linked HA-collagen membrane (HA-collagen ratio of 60/40) was resistant to hyaluronidase digestion and was not cytotoxic to L929 fibroblasts. When implanted into rats for up to 4 weeks it prevented adhesions and was only associated with a mild tissue reaction. The results of this study suggest that the HA-collagen membrane could be a candidate mechanical barrier for preventing postoperative intraperitoneal adhesion.

### EXPERIMENTAL EXAMPLE 2

Materials and Methods

Preparation of Noncrosslinked HA/Collagen Membrane

Membrane A. For Membrane A the final weight ratio of HA/collagen=60/40. HA aqueous solution was prepared from 120 mg hyaluronic acid (sodium salt, MW=1.2×10⁶; LifeCore, Chaska, Minn.) with the use of 7 mL double-distilled water. Collagen (type I/III=85/15; Meddicoll, Sydney, Australia) solution was prepared by dissolving 80 mg collagen in 10 mL 0.5M acetic acid. Three milliliters of 1N NaOH was then slowly added to the collagen solution to adjust the pH to 5.0. The HA and collagen solution were then mixed by a stirrer to form a clear mixture. Ten milliliters of the resulting mixture was then degassed, and cast into a 5×5-cm Teflon dish. The cast solution was allowed to dry in an oven at 35° C.

Membrane B. For Membrane B the final weight ratio of HA/collagen=40/60. The same techniques were applied for the preparation of Membrane B except that the HA/collagen membrane was fabricated with a final weight ratio of HA/collagen=40/60.

Crosslinking of the HA/Collagen Membrane with EDC

The HA/collagen membrane was rinsed several times with 80% acetone and then immersed in 2.5% 1-ethyl-(3-3-dimethylaminopropyl)carbodiimide (EDC) solution (Sigma, St. Louis, Mo.) in 80% acetone (pH=4.7) at 35° C. for 2 h. The crosslinked membrane was washed in 80% acetone for 30 min for 2-3 changes, then in 1M NaCl for 30 min, and finally in RO water for 30 min 4 or 5 times to remove the residual EDC. The washed membrane was then dried, packed, and sterilized under UV overnight before use.

Animal Experimental Grouping

A. preliminary biocompatibility and in vivo degradation study of the HA/collagen membrane was conducted first. Approval was obtained from the Institutional Animal Care and Use Committee prior to the study. All animals received humane care in compliance with the principles of laboratory animal care and use. Crosslinked membranes with known HA/collagen weight ratios (80/20, 60/40, 50/50, and 40/60) were sterilized, rinsed with sterilized PBS, and implanted subcutaneously in the back of adult Sprague-Dawley (SD) rats. After predetermined intervals, the rats were sacrificed and the implanted HA/collagen membrane was excised en bloc together with local tissues. After fixation in 10% formaldehyde solution, embedded in paraffin, sectioned in 6 μm thick, and stained with hematoxylin and eosin, the extent of in vivo degradation and tissue reaction were analyzed.

A rabbit model of laminectomy was then used to assess the efficacy of the crosslinked HA/collagen membranes in the prevention of peridural scar adhesion. Forty-eight adult New-Zealand white rabbits, weighing 2.8-3.2 kg, were used. All rabbits underwent a total laminectomy of the sixth lumbar spine. The rabbits were then randomly divided into three groups. Group I rabbits (n=16) received a weight ratio of HA/collagen=60/40 membrane (Membrane A) onto the exposed dura. Group II rabbits (n=16) received a weight ratio of HA/collagen=40/60 membrane (Membrane B) onto the exposed dura. Group III rabbits (n=16) received no treatment and were used as controls.

Surgical Procedure and Treatment

The surgical procedure was identical to the clinical laminectomy technique. Under general anesthesia, the surgery was performed following strict aseptic technique and meticulous hemostasis. After dissecting the subcutaneous tissue, fascia, and muscles, total laminectomy of the sixth lumbar spine was performed with the use of a powered burr and a lamina punch. The ligamentum flavum was removed, and the underlying dura mater was exposed. After the laminectomy site was well prepared and hemostasis was obtained, the membrane was shaped with scissors to accommodate the defect site. No attempts were made during surgery to prevent displacement of the membrane. The fascia was then closed with 2-0 Vicryl, and the skin was closed with 3-0 nylon sutures. Postoperatively, the rabbits were housed in individual cages, received a normal diet, and were allowed normal activity.

Magnetic Resonance Image Examination

Magnetic resonance image (MRI) examination was performed at 3 months after surgery. After general anesthesia, the MR images were acquired with the use of a 1.5-T wholebody MRI scanner (Magnetom Vision, version VB-33D, Siemens Medical Systems, Germany) with quadrate extremity receiving coil. Four sets of MR images were performed; they were spin echo Ti-weighted images in sagittal (TR=400 ms, TE=12 ms) and transverse (TR=420 ms, TE=15 ms) planes of the animals and gradient recalled echo T2-weighted images in the same locations with spin echo images (TR=400 ms, TE=18 ms, flip angle=20° for sagittal images, and TR=600 ms, TE=25.8 ms, flip angle=30° for transverse images). All MRI studies were performed without contrast enhancement.

Myelogram Examination

Myelogram was performed at 3 months after surgery just prior to euthanasia. After general anesthesia, 1 mL of watersoluble contrast medium (Isovist, Schering AG Pharmaceutical, Berlin, Germany) was intrathecally injected through interspace between the second and third lumbar spines, and anteroposterior (AP) and lateral radiographs of the whole spine were taken.

Histological Examination

Histological examination was performed at 1 month (four rabbits for each group), 2 months (four rabbits for each group), and 3 months after surgery (eight rabbits for each group). The rabbits were euthanized with an intravenous pentobarbital injection. The laminectomy area, including the vertebral column and surrounding tissues, was removed en bloc and fixed in 10% formaldehyde solution. The specimens were decalcified in 5% formic acid and then embedded in paraffin. Seven 6-μm transverse sections in equal distance were obtained from the proximal part to the caudal part of the laminectomy site. Sections were stained with hematoxylin and eosin and examined under a light microscope.

Quantitative histological analyses were performed with the use of a semiautomated image analysis system. This system comprised a microscope (Olympus), a digital camera fixed to the microscope, a computer with image analysis software (Image-Pro Plus, MD), and a high-resolution color monitor. The amount of scar tissue as well as regenerative bone, and extent of adhesion, were measured as described by He et al.(A Quantitative model of post-laminectomy scar formation. Spine 1995; 20:557-563.) Amounts of scar tissue and regenerative bone were expressed in millimeters squared. Extent of adhesion was graded as follows: Grade 0, the dura mater was fiee of scar tissue; Grade 1, only thin fibrosis bands between the scar tissues and the dura mater were observed; Grade 2, continuous adhesion was observed but was less than ⅔ of the laminectomy defect; and Grade 3, scar adhesion was large, more than ⅔ of the laminectomy defect, and/or extended to the nerve roots.

Statistics

Statistical analysis was performed with use of the SPSS for windows statistical package (version, 10.0; SPSS, Chicago, Ill., 1999). Comparisons of amounts of scar tissue and regenerative bone were performed with the use of analysis of variance (ANOVA) among groups and also among different time periods for the same group. When a significant difference was round, a post hoc analysis was done to determine which specific difference was significant. A two-tailed nonparametric Kruskal-Wallis test was used to determine statistical significance of the extent of peridural adhesion among groups and also among different time periods for the same group. Significance was defined as p<0.05.

Results

Biocompatibility and In Vivo Degradation

Subcutaneous implantation of the HA-collagen membrane elicited minimal inflammatory reaction. All the membranes showed good biocompatibility, as only a few polymorphonuclear cells infiltrated into the samples for the first 4-7 days after implantation. With a higher final weight ratio of HA (HA/collagen=80/20), the membrane firmly attached to the surrounding tissues once they were applied onto the tissues. They were liquefied, became gel-like, and were biodegraded within a few days in vivo. With decreasing final weight ratio of HA (HA/collagen=40/60), the membrane remained in tissues. for about 1 month after implantation. According to the preliminary in vivo study, final weight ratios of HA/collagen=60/40 as well as 40/60 membranes were chosen to be tested for the prevention of peridural adhesion because they retained in tissues for certain time period to act as a mechanical barrier and elicited none or only minimal tissue reaction in vivo.

General Observation

The laminectomy sites were uniform in size for all animals, measuring approximately 1.4 cm long and 0.5 cm wide. Two animals suffered from moderate neurological deficit after surgery and were dropped from the study. Active bleeding during surgery was encountered in four rabbits. The bleeding was controlled by compression and stopped before wound closure. These four rabbits were not included in further analysis. However, histological examination was performed. The remaining animals were healthy and ambulatory without evidence of neurological deficit postoperatively. No clinical signs of infection or hematoma were encountered, and all wounds healed uneventfully after surgery.

MR Image and Myelogram Examination

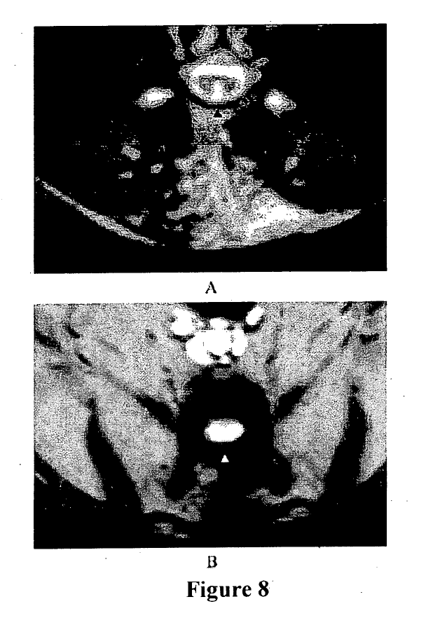

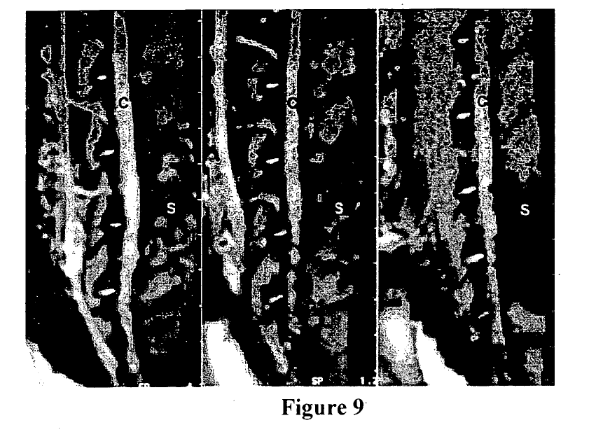

Myelogram was inadequate for evaluating postoperative peridural scar adhesion. None of the animals showed extradural spinal cord compression at the level of laminectomy or stenotic changes of the intrathecal volume. Axial MR images obtained at the laminectomy level of Membrane B-treated animals showed discrete hyposignal space between the dura and the surrounding scar tissues [FIG. 8(A)]. Adhesions were not observed over the dura. Continuity between the surrounding scar tissues and the dura suggesting the existence of peridural scar adhesions was found at laminectomy sites of control rabbits [FIG. 8(B)]. Sagittal MRI also showed extensive scar formation with the scar tissues abutting the spinal canal in controls, and fewer scars in the experimental rabbits [FIGS. 9(A-C)].

Qualitative Histological Evaluation

No acute inflammatory reaction was found in all groups. Minimal to mild chronic inflammatory response consisting of macrophages and monocytes was observed adjacent to the Membrane A at 2-3 months, histological sections. There was neither a humoral nor a cell-mediated immune response (no plasma cells, eosinophils, and few lymphocytes). All HA/collagen membranes were visible at 1 month's histological sections. All A membranes and half of the B membranes were still visible at 2 months' histological sections. All B membranes were not visible (biodegraded) at 3 months' sections, whereas half of the A membranes were still visible at 3 months' sections.

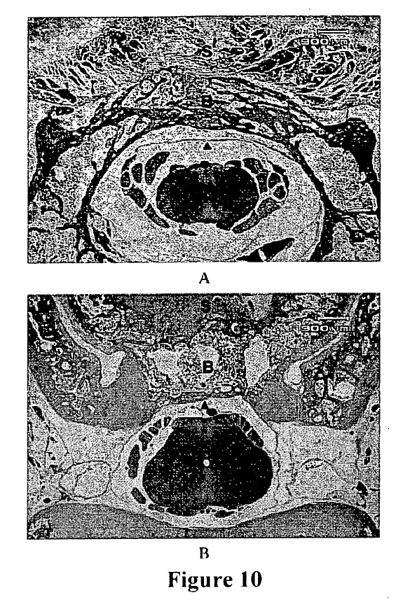

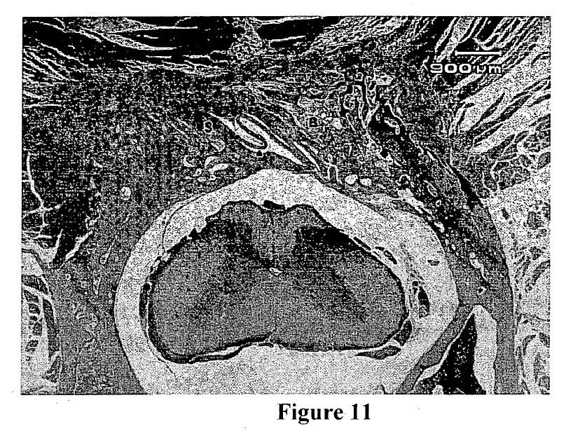

Bony regeneration at the laminectomy window was noted. The laminectomy window decreased in size due to bone growth extending from the periphery of the defect. Histological evaluation confirmed that scar tissues were present at all laminectomy sites of all animals. The amount of scar tissue tended to decrease with time in all three groups. At 3 months after surgery, an identifiable plane existed between the regenerated bone and the dura at the Membrane B-treated laminectomy site, and the scar tissues above the regenerated bone were scanty and replaced by fibrofatty tissues [FIG. 10(A)]. However, the defect sites of the control animals demonstrated a moderate amount of scar tissue with peridural scar adhesion [FIG. 10(B)]. Some of the membranes had shifted slightly, exposing a space for scar tissues to come close to the peridural space (FIG. 11).

Quantitative Histological Evaluation

The results of quantitative histological analysis are shown in Table 11.

TABLE 11
Results of Quantitative Histological Analysis

| | | |
|---|---|---|
| Membrane A | Membrane B | Control |

Amount of scar tissues (mm²)

| | | | |
|---|---|---|---|
| 1 month | 20.3 ± 4.9 | 10.6 ± 2.7 | 29.1 ± 5.1 |
| 2 months | 10.5 ± 2.8 | 4.8 ± 2.6 | 21.9 ± 6.4 |
| 3 months | 7.1 ± 2.3 | 3.2 ± 2.6 | 11.1 ± 3.7 |

Amount of regenerative bone
(mm²)

| | | | |
|---|---|---|---|
| 1 month | 4.33 ± 0.95 | 5.38 ± 0.76 | 0.88 ± 0.28 |
| 2 months | 5.95 ± 0.71 | 6.53 ± 0.74 | 1.08 ± 0.35 |
| 3 months | 6.98 ± 0.69 | 7.06 ± 0.91 | 3.99 ± 0.89 |

Extent of adhesion^{@}

| | | | |
|---|---|---|---|
| 1 month | 0:0:2:2 | 0:0:2:2 | 0:0:1:3 |
| 2 months | 0:0:2:2 | 1:2:1:0 | 0:0:1:3 |
| 3 months | 2:2:2:0 | 4:1:1:0 | 1:1:3:1 |

^{@}Number expressed as Grade 0:Grade 1:Grade 2:Grade 3.

Amount of Scar Tissue.

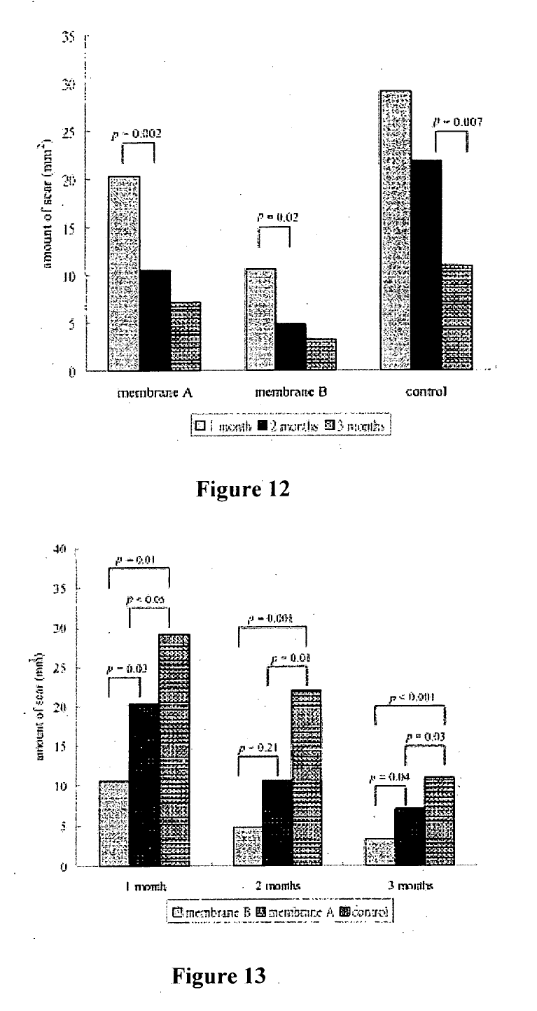

The amount of scar tissue tends to decrease with time in all three groups whether the laminectomy site received treatment or not (FIG. 12). The amount of scar tissue decreased more quickly in the membrane-treated sites than in the controls. Significant decrease in amount of scar tissues was noted in the 2 months' sections at laminectomy sites treated with HA/collagen membranes, whereas significant decrease in amount of scar tissues was noted only at 3 months' sections of the control. When amounts of scar tissue at the same time period are compared, a significantly less scar tissue was noted at laminectomy sites receiving either membrane treatment than at the control at all three time periods (FIG. 13).

Amount of Regenerative Bone. The laminectomy sites decreased in size due to bone growth extending from the periphery of the defect. The lamiiiectomy defect treated with either HA/collagen membrane exhibited significantly greater amount of regenerative bone than that of the control at all time periods (p<0.001, respectively).

Extent of Peridural Scar Adhesion.

Extent of peridural scar adhesion was significantly reduced at laminectomy sites treated with Membrane B when compared with that of the control at 3 months after surgery (p<0.04). Laminectomy sites treated with Membrane A showed more peridural scar adhesion than the sites treated with Membrane B, and less peridural scar adhesion than the controls, but none of the differences was statistically significant. At other time periods, the extent of peridural scar adhesion showed the same trend as displayed at the 3 months' histological sections. However, none of the groups at other time periods showed significant difference.

Discussion

The study shows that the EDC-crosslinked HA-collagen membrane is an effective and safe antiscar adhesion material that can be applied in vivo without causing significant adverse reaction in an adult rabbit laminectomy model. The crosslinked HA/collagen membrane undergoes bioresorption, conforms to anatomical topography, and becomes a physical barrier intended to prevent peridural adhesions following laminectomy. Treatment with the EDC-crosslinked HA-collagen membrane does not affect healing of the skin, the subcutaneous tissue, or the muscle, but primarily prevents scar formation and adhesion adjacent to the dura at the laminectomy site. The final weight ratio of HA/collagen=40/60 membrane shows better antiadhesion effect than the final weight ratio of 60/40 membrane, and demonstrates significant effect on reducing extent of peridural adhesion compared to the control.

Though the relationship between clinical symptoms and scar or adhesion formation is debatable, as many as 24% of all failed back surgery syndrome cases are attributed to peridural scar adhesion. The scar tissues may restrict nerve root motility, making it susceptible to even small recurrent disc herniation or stenosis. The amount of scar and adhesion formation should be as minimal as possible. The exact mechanism of peridural scar adhesion is not clear. Key and Ford proposed that the annulus fibrosus was the source of post-laminectomy scar tissue. LaRocca and McNabb suggested that fibrosis was caused by a posterior invasion of fibroblasts from the erector spinal muscles forming a membrane called the laminectomy membrane. Songer et al. hypothesized that epidural fibrosis occurred when epidural fat was replaced by hematoma, formed in the path of surgical dissection. The hematoma was absorbed and replaced by granulation tissues, which matured into dense fibrotic tissues. Regardless of the exact mechanism of peridural scar adhesion, it seems that the key feature affecting the extent of peridural adhesion is to prevent or limit fibroblasts from contacting the exposed dura in the early healing phase.

An ideal antiadhesive barrier film should be effective, biocompatible, easily applicable, and absorbable, but remain in place for certain period. The biocompatibility of HA and collagen in vivo as well as adjacent to the dura and peripheral nerve tissues has been documented in several previous studies. Hyaluronic acid has been reported, to decrease adhesion formation in areas such as around traumatized tendons, in the intraperitoneal cavity, and in strabismus surgery. Collagen-based membranes or gel after modification have been used as adhesion barriers. The use of collagen as the base of the composite provides good mechanical properties for handling. EDC does not chemically bind to polysaccharide molecules, and makes the final product soft and easily applicable at any anatomical site. HA residence time was reported to be important in the prevention of postsurgical adhesion. Welch et al. suggested that the materials should be present and maintain their properties beyond the critical healing time of 6-12 weeks. Crosslinking of HA/collagen membrane is effective in prolonging the residence time of hyaluronic acid. Preliminary study of the HA-collagen membrane showed that these membranes formed a thin, white, nearly transparent layer that prevented fibroblasts from making contact with the exposed tissues. Very few inflammatory cells and chronic reaction were observed within the scar tissues of all treated animals when the final weight ratio of collagen did not exceed 60%. There was no increase in the incidence of wound dehiscence, hematoma, or infection at the implantation sites. With the higher final weight ratio of HA, the membrane turned to gel very quickly because of hydration. The membranes remained in tissues as the final weight ratio of collagen increased. For adhesion prevention, it was hypothesized that the membrane should remain in place long enough to act as a barrier, but without causing tissue reactions. The final weight ratios of HA/collagen=60/40 and 40/60 membranes fitted the above-mentioned criteria better, and were thus chosen to be tested in the laminectomy model.

The EDC-crosslinked HA-collagen membrane was found to be an effective barrier for peridural scar adhesion in this study. The HA-collagen membrane formed a physical barrier that effectively inhibited the formation of peridural scar adhesions. This membrane also formed guided bone regeneration over it, and thus prevented fibroblasts from making contact with the exposed dura and nerve roots. No adhesions were found between the regenerated bone and the dura. The HA-collagen membrane-treated groups demonstrated less peridural scar adhesion when compared to the untreated controls. It was further observed that once the barrier film was reached through the dense posterior scar, a distinguishable dissection plane was present. Statistical analysis demonstrated that the final weight ratio of 40/60 HA-collagen membranes significantly reduced peridural scar adhesion at 3 months after surgery when compared to the controls.

New bone formation from the margins of laminectomy defect was found as early as 30 days postoperatively. There was more regenerative bone in the HA-collagen membrane-treated group than in the control group. Jacob et al. observed that when MeroGel hyaluronic acid (Medtronic Xomed Surgical Products, Jacksonville, Fla.) was placed adjacent to traumatized remodeling bone, it might have osteogenic potential. Liu et al. also reported that implantation of collagen-HA matrix demonstrated good biocompatibility and exhibited greater osteoconductive potential than matrices composed of either crosslinked collagen or hyaluronic acid alone. Because the lamina is seldom implicated as the actual cause of spinal compression, osseous healing of a laminectomy defect may be desirable. However, it is uncertain whether significant neolaminization will occur in the human spine. The bone regeneration demonstrated in this study might not be indicative of a similar response in clinical setting because of species differences in osteogenic potential.

One problem related to previously studied barrier film includes marginal scar invasion due to poor fit of the material to the laminectomy defect. Welch et al. found that translation of the barrier film allowed scar tissues to migrate underneath into the epidural space. No attempts were made during surgery to prevent this displacement in this study. The membrane was observed to be folded in some histological sections, which allowed scar tissues to migrate underneath into the epidural space. When the membrane was displaced, areas demonstrated moderate scar formation. Despite displacement of the barrier, approximately 70% to 80% of the exposed dural surface remained in contact with the membrane. Techniques or methods to ensure secure attachment of the membrane in the proper position may improve the anti-adhesion effectiveness.

Active bleeding was encountered in four rabbits at the time of surgery. The bleeding was controlled by compression. The bleeding stopped before wound closure. These four rabbits were not included in further analysis. However, histological sections showed extensive peridural scar adhesion whether the laminectomy sites were treated with the HA/collagen membrane or not. A highly significant peridural scar adhesion was noted when active bleeding was noted compared to the membrane-treated groups (p<0.001, respectively) and the controls (p=0.02). This complication appeared to overwhelm any subsequent effect of interpositional membrane. Techniques to prevent bleeding and hematoma formation by meticulous atraumatic dissection are of the utmost importance in reducing scar formation and peridural adhesion.

The methods of the current study used quantitative histological analysis to assess differences in scar formation and peridural scar adhesion. Attempt to quantify scar formation histologically was tedious because of the number of sections required. With the use of semiautomatic analysis, surface measurements including scar tissue area and regenerative bone area were easily performed and there was a very good interobserver correlation. Determination of cell density was time consuming and interobserver correlation was poor because classification and counting were difficult with a semiautomatic procedure and variation in different section areas. The study did not perform a discectomy. Thus, the effects of the crosslinked HA-collagen membrane might have on scar formation at a discectomy site and on annular ligament healing could not be assessed. The study suggested that compared to the nontreated group, the EDC-crosslinked HA-collagen membrane decreased peridural scar adhesion in the rabbit laminectomy model. Whether this result has any clinical significance in humans remains to be proven in further studies.

Although the invention has been described with reference to the presently preferred embodiments, it should be understood that various modifications can be made by those skilled in the art without departing from the invention. Accordingly, the scope of the present is limited by the following claims.

1. A method for producing cross-linked polysaccharide-protein bio-composites, comprising the steps of:
(a) preparing a mixture of a polysaccharide and a protein in a solution at a weight ratio of polysaccharide to protein in a range of 20/80 to 80/20;
(b) adjusting the mixture at a pH value between 3 and 11 by adding either an acid or a hydroxide, forming into a matrix having a desired shape;
(c) subjecting the matrix to cross-linking reaction by using a cross-linking agent in a mixture of water and one or more organic solvents.

2. The method of claim 1, wherein the polysaccharide in the step (a) is selected from the group consisting of hyaluronic acid, carboxymethyl cellulose, pectin, starch, chondroitin-4-sulfate, chondroitin-6-sulfate, alginate, chitosan, agar, carragenan, and guar gum.

3. The method of claim 1, wherein the protein in the step (b) is selected from the group consisting of collagen, gelatin, or a mixture thereof.

4. The method of claim 1, wherein the acid used in the step (b) is selected from the group consisting of acetic acid, hydrochloric acid, or a mixture thereof.

5. The method of claim 1, wherein the hydroxides used in the step (b) is selected from the group consisting of sodium hydroxide, potassium hydroxide, or a mixture thereof.

6. The method of claim 1, wherein the protein solution is prepared as an acid solution and the polysaccharide solution is prepared as an alkali solution, respectively.

7. The method of claim 1, wherein the protein solution is a collagen solution prepared as an alkali solution and the polysaccharide is prepared as an acid solution so that pH of the resultant mixture is in a range between 5 and 11.

8. The method of claim 1, wherein the protein solution is a collagen solution prepared as an acid solution and the polysaccharide solution is prepared as an alkali solution so that pH of the resultant mixture is in a range between 5 and 11.

9. The method of claim 1, wherein the protein solution is a gelatin solution in de-ionized water, and the ion strength is adjusted to a desired strength by adding sodium chloride.

10. The method of claim 1, wherein the matrix having a desired shape in the step (b) is a porous film matrix formed by casting the degassed matrix into a film and drying in an oven at a temperature of from 20° C. to 45° C.

11. The method of claim 1, wherein the matrix having a desired shape in the step (b) is a porous matrix formed by freezing the degassed matrix in a refrigerator at a temperature of from −30° C. to −100° C. and then pore-forming to give the porous matrix having a porous structure which is inter-connective.

12. The method of claim 11, wherein the pore-forming procedure is carried by at least one method selected from the group consisting of (1) freeze-drying method, (2) supercritical CO₂ foaming method, (3) phase immersing method, (4) critical point drying method, (5) fiber meshes method, (6) membrane lamination, and (7) particulates leaching method.

13. The method of claim 1, wherein the matrix in step (b) is a power matrix formed by dropping the degassed matrix into a freezing solution at a temperature of from −30° C. to −100° C. by using a syringe, and pore-forming to give the powder matrix.

14. The method of claim 13, wherein the pore-forming procedure is carried by at least one method selected from the group consisting of (1) freeze-drying method, (2) supercritical CO₂ foaming method, (3) phase immersing method, (4) critical point drying method, and (7) particulates leaching method.

15. The method of claim 1, wherein the matrix in step (b) is a fiber matrix formed by squeezing the degassed matrix into a solution of a coagulant in a mixture of water and an organic solvent, and pore-forming to give a fibrous matrix having a thickness of from 50 um to 1 mm.

16. The method of claim 15, wherein the pore-forming procedure is carried by at least one method selected from the group consisting of (1) freeze-drying method, (2) supercritical CO₂ foaming method, (3) phase immersing method, (4) critical point drying method, (5) fiber meshes method, (6) membrane lamination, and (7) particulates leaching method.

17. The method of claim 16, wherein the organic solvent is chosen from the group consisting of 1,4-dioxane, chloroform, methylene chloride, N, N-dimethylfomiamide, N,N-dimethylacetamide, ethyl acetate, acetone, methyl ethyl ketone, methanol, ethanol, propanol, isopropanol, butanol and a mixture thereof; a percentage of the organic solvent is from 60% to 100% based on the total weight of the mixture of water and the organic solvent.

18. The method of claim 17, wherein the organic solvent is a mixture of ketones and alcohols, and the percentage of the organic solvent is from 75% to 100% based on the total weight of the mixture of water and the organic solvent.

19. The method of claim 1, wherein the cross-linking agent in step (c) is a carbodiimide.

20. The method of claim 19, wherein the carbodiimide is selected from the group consisting of 1-methyl-3- (3-dimethylaminopropyl)-carbodiimide, 3-(3- dimethylaminopropyl)-3-ethyl-carbodiimide, 1-ethyl-3- (3-dimethylaminopropyl)-carbodiimide and a mixture thereof.

21. The method of claim 1, wherein the mixture of water and organic solvent in the step (c) is consisting of 5%-50% by weight of water and 95 to 50% by weight of either ethanol or acetone, or the both; and the cross-linking reaction is carried out by using 0.5 to 25% by weight of carbodiimide under a pH of 4-5.5 at a temperature of from 20° C.-45° C. for 1-6 hrs.

22. The method of claim 21, wherein the mixture of water and organic solvent in the step (c) is consisting of 5%-30% by weight of water and 95 to 70% by weight of either ethanol or acetone, or the both; and the cross-linking reaction is carried out by using 1 to 5% by weight of carbodiimide under a pH of 4˜5.5 for 2-4 hrs.

23. The method of claim 1, which, after the step (c), further comprises a step of washing the composite with a mixture of water and organic solvent, immersing it in a salt solution, and then washing it with distilled water.

24. The method of claim 23, wherein the mixture of water and organic solvent is consisting of 5%-50% by weight of water and 95 to 50% by weight of either ethanol or acetone, or the both, and the immersion time is from 0.5-3 hrs.

25. The method of claim 24, wherein the mixture of water and organic solvent is consisting of 5%-30% by weight of water and 95 to 70% by weight of either ethanol or acetone, or the both.

26. The method of claim 23, wherein the salt solution is used in a concentration of 0.15-4M and the salt used is chosen from the group consisting of sodium chloride, dibasic sodium phosphate and a mixture thereof.

27. A bio-composite consisting of polysaccharide and protein through a crosslinking agent, in which a weight ratio of polysaccharide to protein in a range of 20/80 to 80/20.

28. The bio-composite of claim 27, which is prepared by the method of any one of claims 1-26.

29. A use of the bio-composite of claim 27 in prevention or reduction of post-surgical adhesion.

30. A use of the bio-composite of claim 27 in bone regeneration.
